(19) United States
(12) Patent Application Publication
(10) Pub. No.: US 2004/0202730 A1
(43) Pub. Date: Oct. 14, 2004

(54) Rosmarinic acid composition

(76) Inventors: Robert Gow, Naples, FL (US); John Pierce, Moreno Valley, CA (US); Brian Pierce, Moreno Valley, CA (US); William Birdsall, Naples, FL (US)

Correspondence Address: Hillary W. Hawkins, Esquire - Williams Mullen, Two James Center, 1021 East Cary Street, Richmond, VA, 23219, US

(21) Appl. No.: 10/407,685

(22) Filed: Apr. 8, 2003

(51) Int. Cl. A61K 35/78; A61K 31/704

(52) U.S. Cl. 424/728; 424/745; 514/33

(57) ABSTRACT

This invention is related to an improved composition for treating heart conditions. More specifically, the present invention is related to an improved composition for treating heart conditions containing borneol, rosmarinic acid and a ginsenoside selected from the group consisting of ginsenoside Rg1 and ginsenoside Rb1.

### FIELD OF THE INVENTION

This invention is related to an improved composition for treating heart conditions. More specifically, the present invention is related to an improved composition for treating heart conditions containing borneol, rosmarinic acid and a ginsenoside selected from the group consisting of ginsenoside Rg1 and ginsenoside Rb1.

### BACKGROUND

It has long been know that Rosemary (Rosmarinus officinalis) is a beneficial medicinal plant and contains potent antioxidants such as rosmarinic acid, as well as essetional oils, such as borneol.

Ginseng has also been recognized as an important herbal medicine. There are many varieties of ginseng and each variety of the ginseng plant contains many pharmacologically active components. Correctly chosen mixtures of such components often have unexpected beneficial effects.

Heretofore, there has been no disclosure of a composition containing rosmarinic acid, borneol and a ginsenoside selected from the group consisting of ginsenoside Rg1 and ginsenoside Rb1 and its pharmacological activity in the treatment of heart conditions.

It would be advantageous and is an object of the present invention to provide a product containing rosmarinic acid, borneol and a ginsenoside selected from the group consisting of ginsenoside Rg1 and ginsenoside Rb1.

Products of the present invention may be used as dietary supplements, e.g., mixed in drinks or packaged in capsules. It would also be advantageous to produce a dry flowable powder of the aforementioned product. The powder can be distributed as is or further processed, e.g., to produce tablets.

It would also be advantageous to deliver the present composition in the form of a rapid-dissolve tablet which would permit its active components to be absorbed orally.

### SUMMARY OF THE INVENTION(S)

These and other objectives are met by use of a product, such as a paste, resin, oil, or a powder suitable for use in a fast dissolve tablet and other applications, containing rosmarinic acid, borneol and a ginsenoside selected from the group consisting of ginsenoside Rg1 and ginsenoside Rb1. The resulting product has the unexpected benefit of reducing heart conditions. Specifically, the present combination helps make red blood cells more elastic, prevents atherosclerosis, improves contractile ability of the heart, lowers blood pressure, inhibits lipid oxidation, ameliorates the effects of ischemia, improves blood circulation, resolves blood stasis, inhibits platelet aggregation, and prevents restenosis.

The composition of the present invention can originally be in the form of a paste, resin, or oil and then processed for various uses. For direct ingestion, for example, a paste can be sweetened and flavored. In addition or alternatively, the paste can be mixed with other dietary supplements, such as the very sweet tasting herb Stevia rebaudiana, flavors, anti-oxidants, and/or extract of Hex paraguariensis, as well as other botantical extracts.

A paste can also be further processed to prepare a high quality dry flowable powder which can be used, for example, to produce an ingestible tablet. In one embodiment, a paste is combined with a carrier, such as maltodextrin, dextrose, or starches and mixed with a suitable solvent, such as ethyl alcohol or water. The mixture is then spray dried to produce a powder having grains comprising rosmarinic acid, borneol and ginsenoside Rg1 and/or ginsenoside Rb1 and the carrier. In a second embodiment, an emulsion of paste is formed in water or ethyl alcohol using, e.g., magnesium carbonate, magnesium carbonate+silica (at up to about 2% by weight), whey protein, maltodextrin, carboxymethylcellulose and/or other suitable materials. The emulsion is then dried and powdered.

The resulting powder can then be formed into a tablet that, when placed in the mouth, dissolves rapidly over a period of between about 5 seconds to about 120 seconds and preferably in about 15 to about 60 seconds. A tableting powder can be formed by combining between about 18% to about 60% by weight of the powdered composition with between about 30% to about 80% by weight of a dry water-dispersible adsorbant such as magnesium carbonate, or a diluent, such as lactose. Other dry tablet additives, such as one or more of a sweetener, flavoring and/or coloring agents, a binder, such as acacia or gum arabic, a lubricant, a disintegrant, and a buffer, can also be added to the tableting powder. Preferably, the dry ingredients are screened to a particle size of between about 80 to about 100 mesh.

### DESCRIPTION OF THE PREFERRED EMBODIMENT(S)

The present composition contains rosmarinic acid, borneol and a ginsenoside selected from the group consisting of ginsenoside Rg1 and ginsenoside Rb1. In a preferred embodiment, the borneol component comprises about 0.05% to about 85% of the sum of the masses of the three components, more preferably, between about 10% to about 65%. The rosmarinic acid component may comprise about 0.05% to about 85% of the sum of the masses of the three components, more preferably, between about 10% to about 65%. The ginsenoside component may comprise about 0.05% to about 85% of the sum of the masses of the three components, more preferably, between about 10% to about 65%.

According to an aspect of the invention, a paste of the present invention can be further processed to produce a dry, flowable powder. The powder can be used as a dietary supplement that can be added to various edible products. The powder is also suited for use in a rapid dissolve tablet.

According to a particular aspect of the invention, the powder is produced to have a composition that is particularly well suited for delivery in the oral cavity of human subjects, e.g., via a rapid dissolve tablet.

In one embodiment of a method for producing the powder, the paste is mixed with a suitable solvent, such as ethyl alcohol or water, along with a suitable food-grade carrier material, such as maltodextrin, dextrose, or starch and the mixture is spray air-dried using conventional techniques to produce a powder having grains of very small particles combined with the food-grade carrier material.

A wide variety of tablet formulations can be made. Preferably, the tablet has a formulation that results in a rapid dissolution or disintegration in the oral cavity. The tablet is preferably of a homogeneous composition that dissolves or disintegrates rapidly in the oral cavity to release the over a period of about 5 seconds or less to about 120 seconds or more, preferably about 15 to about 60 seconds.

Various rapid-dissolve tablet formulations known in the art can be used. Representative formulations are disclosed in U.S. Pat. Nos. 5,464,632, 6,106,861, and 6,221,392, the entire contents of which are expressly incorporated by reference herein. A particularly preferred tableting composition or powder contains about 10% to about 60% by weight of the powder and about 30% to about 60% of a water-soluble diluent. Suitable diluents include lactose, dextrose, sucrose, mannitol, and other similar compositions. Lactose is a preferred diluent but mannitol adds a pleasant, cooling sensation and additional sweetness in the mouth. More than one diluent can be used. A sweetener can also be included, preferably in an amount of between about 3% to about 40% by weight depending on the desired sweetness. Preferred sweetening substances include sugar, saccharin, sodium cyclamate, aspartame, and Stevia extract, used singly or in combination, although other sweeteners could alternatively be used. Flavorings, such as mint, cinnamon, citrus (e.g., lemon or orange), can also be included, preferably in an amount between about 0.001% to about 4% by weight.

Typically, this tableting composition will maintain its form without the use of a binder. However, if needed, various binders are suitable and can be added in an amount of between about 5% to about 15% or as necessary. Preferred binders are acacia or gum arabic. Alternative binders include sodium alginate, extract of Irish moss, panwar gum, ghatti gum, mucilage of isapol husks, carboxymethylcellulose, hydroxyethylcellulose, methylcellulose, polyvinylpyrrolidone, VEEGUM® (available from R.T Vanderbilt Co., Inc. of Norwalk, Conn.), larch arabogalactan, gelatin, Kappa carrageenan, copolymers of maleic anhydride with ethylene or vinyl methyl ether.

A tablet according to this aspect of this invention typically does not require a lubricant to improve the flow of the powder for tablet manufacturing. However, if it is so desired, preferred lubricants include talc, magnesium stearate, calcium stearate, stearic acid, hydrogenated vegetable oils, and carbowax in amounts of between about 2% to about 10% by weight.

Similarly, a disintegrant is not expected to be necessary to produce rapid dissolve tablets using the present tablet composition. However, a disintegrant can be included to increase the speed with which a resulting tablet dissolves in the mouth. If desired, between about 0.5% to about 1% by weight of a disintegrant can be added. Preferred disintegrants include starches, clays, celluloses, algins, gums, crosslinked polymers (including croscarmelose, crospovidone and sodium starch glycolate), VEEGUM® HV, agar, bentonite, natural sponge, cation exchange resins, aliginic acid, guar gum, citrus pulp, sodium lauryl sulphate in an amount of about 0.5% to about 1% of the total mass of the tablet.

It is also generally considered unnecessary to buffer the tablet composition. However, a buffer may be beneficial in specific formulations. Preferred buffering agents include mono- and di-sodium phosphates and borates, basic magnesium carbonate and combinations of magnesium and aluminum hydroxide.

In a preferred implementation, the tableting powder is made by mixing in a dry powdered form the various components as described above, e.g., active ingredients (rosmarinic acid, borneol and ginsenoside Rg1/Rb1), diluent, sweetening additive, and flavoring, etc. An overage in the range of about 10% to about 15% of the active extract of the active ingredient can be added to compensate for losses during subsequent tablet processing. The mixture is then sifted through a sieve with a mesh size preferably in the range of about 80 mesh to about 100 mesh to ensure a generally uniform composition of particles.

The tablet can be of any desired size, shape, weight, or consistency. The total weight of the active components in the form of a dry flowable powder in a single oral dosage is typically in the range of about 80 mg to about 600 mg. An important consideration is that the tablet is intended to dissolve in the mouth and should therefore not be of a shape that encourages the tablet to be swallowed. The larger the tablet, the less it is likely to be accidentally swallowed, but the longer it will take to dissolve or disintegrate. In a preferred form, the tablet is a disk or wafer of about ⅛ inch to about ¾ inch in diameter and about 0.2 inch to 0.08 inch in thickness, and has a weight of between about 160 mg to about 1,200 mg. In addition to disk, wafer or coin shapes, the tablet can be in the form of a cylinder, sphere, cube, or other shapes.

We claim:

1. A composition comprising an active component, said active component comprising a rosmarinic acid component, a borneol component and a ginsenoside component, wherein said ginsenoside component is selected from the group consisting of ginsenoside Rb1 and ginsenoside Rg1.

2. The composition of claim 1 wherein said rosmarinic acid component is between about 0.05% to about 85%, by mass, of said active component.

3. The composition of claim 1 wherein said borneol component is between about 0.05% to about 85%, by mass, of said active component.

4. The composition of claim 1 wherein said ginsenoside component is between about 0.05% to about 85%, by mass, of said active component.

5. The composition of claim 1 wherein said ginsenoside is ginsenoside Rb1.

6. A dry flowable powder having the composition of claim 1.

7. An ingestible product comprising the composition of claim 1.

8. The product of claim 7, further comprising extract of stevia.

9. The composition of claim 1, wherein the material is a paste extract containing the active component.

10. The composition of claim 1, wherein the material is a liquid extract containing the active component.

11. The composition of claim 1, wherein the material is an oil containing the active component.

12. The composition of claim 1, wherein the material is a powder containing the active component.

13. The composition of claim 12, further comprising a water-dispersible adsorbant.

14. The composition of claim 13, wherein the water-dispersible adsorbant is magnesium carbonate.

15. The powder of claim 6, for use as a tableting powder.

16. The powder of claim 6, further comprising a lubricant.

17. The powder of claim 16, wherein the lubricant is selected from a group consisting of talc, magnesium stearate, calcium stearate, stearic acid, hydrogenated vegetable oil, and carbowax.

18. The powder of claim 6 for use as a capsule filling powder.

19. The powder of claim 6, further comprising between about 3% and about 40% by weight of a sweetener.

20. The powder of claim 19, wherein the sweetener is selected from a group consisting of sugar, saccharin, sodium cyclamate, aspartame, and extract of stevia.

21. The powder of claim 6, further comprising between about 0.001% and about 4% by weight of a flavoring agent.

22. The powder of claim 21, further comprising about 0.5% to about 2% by weight of a coloring agent.

23. The powder of claim 6, further comprising about 5% to 15% by weight of a binder.

24. The powder of claim 23, wherein the binder is selected from the group consisting of acacia, gum arabic, sodium alginate, extract of Irish moss, panwar gum, ghatti gum, mucilage of isapol husks, carboxymethylcellulose, hydroxyethylcellulose, methylcellulose, polyvinylpyrrolidone, Veegum, larch arabogalactan, gelatin, carrageenan, and copolymers of maleic anhydride and ethylene and/or vinyl methyl ether.

25. The powder of claim 6, further comprising about 0.5% to 1% by weight of a disintegrant.

26. The powder of claim 25, wherein the disintegrant is selected from a group comprising starch, clay, cellulose, algin, gum, crosslinked polymers, Veegum, agar, bentonite, natural spone, cation exchange resins, aliginic acid, guar gum, citrus pulp, and sodium lauryl sulphate.

27. The powder of claim 6, further comprising a buffer.

28. The powder of claim 27, wherein the buffer is selected from a group consisting of mono- and di-sodium phosphates and borates, magnesium carbonate, and a combination of magnesium hydroxide and aluminum hydroxide.

29. The powder of claim 6, further comprising a diluent.

30. The powder of claim 29, wherein the diluent is selected from a group consisting of lactose, dextrose, sucrose, and mannitol.

31. A tablet formed from the powder of claim 6.

32. A capsule filled with the powder of claim 6.

33. The tablet of claim 32, wherein the extract is present in a weight of between about 80 mg to about 600 mg.

34. A rapid dissolve tablet comprising: (a) between about 18% to about 60% by weight of the powder of claim 6; (b) between about 30% to about 80% by weight of a water-soluble diluent; (c) between about 5% to about 15% by weight of a binder; and (d) between about 3% to about 40% by weight of a sweetener, with the percentages being by weight and totaling 100%.

35. The tablet of claim 34, wherein the water-soluble diluent is selected from the group consisting of lactose, dextrose, sucrose, and mannitol; wherein the binder is selected from the group consisting of acacia, gum arabic,,sodium alginate, extract of Irish moss, panwar gum, ghatti fum, mucilage of isapol husks, carboxymethylcellulose, hydroxyethylcellulose, methylcellulose, polyvinylpyrrolidone, Veegum, larch arabogalactan, gelatin, carrageenan, and copolymers of maleic anhydride with ethylene and/or vinyl methyl ether; and wherein the sweetener is selected from the group consisting of sugar, saccharin, sodium cyclamate, aspartame, and extract of stevia.

36. The tablet of claim 34, wherein the tablet dissolves orally in between about 5 seconds and about 120 seconds.

37. The composition of claim 1 wherein said ginsenoside is ginsenoside Rg1.
